(19) United States
(12) Patent Application Publication
(10) Pub. No.: US 2022/0249822 A1
(43) Pub. Date: Aug. 11, 2022

(54) Applicator for Intradermal Microneedle Patches

(71) Applicant: Tokitae, LLC, Bellevue, WA (US)

(72) Inventors: Ari Karchin, Seattle, WA (US); John Arthur Ohrt, Spokane, WA (US); Evan Schuster, Seattle, WA (US); David J. Yager, Carnation, WA (US); Brandon Gregory Folk, Hayden, ID (US)

(73) Assignee: Tokitae, LLC, Bellevue, WA (US)

(21) Appl. No.: 17/670,048

(22) Filed: Feb. 11, 2022

Related U.S. Application Data
(60) Provisional application No. 63/148,460, filed on Feb. 11, 2021.

(51) Int. Cl. A61M 37/00

(52) U.S. Cl. CPC A61M 37/0015; A61M 2037/0061; A61M 2037/0023

(57) ABSTRACT

The present disclosure relates to applicators for microneedle array patches and methods of application of microneedle array patches to a skin surface. The applicator can include a shaft having an opening at a first end for affixing a microneedle array patch, a plunger positioned substantially within the shaft, a loading mechanism operably associated with the plunger within the shaft, and a force applicator. The method can include affixing the microneedle array patch to a first end of a plunger of a microneedle array patch applicator, loading the plunger and the affixed microneedle array patch to a loaded position, positioning the opening against the skin region of the individual and operating the force applicator to release the plunger.

### CROSS-REFERENCE TO RELATED APPLICATIONS

This application claims the benefit to provisional U.S. Patent Application No. 63/148,460, titled “APPLICATOR FOR INTRADERMAL MICRONEEDLE ARRAY PATCHES” and listing Ari Karchin as first inventor, filed Feb. 11, 2021. The entire contents of the above-referenced application and of all priority documents referenced in the Application Data Sheet filed herewith are incorporated by reference herein, in their entireties, for all purposes.

### FIELD

This disclosure is directed to improved devices and methods of applicators for intradermal microneedle array patches.

### BACKGROUND

Therapeutic agents such as vaccines require a means of delivery through the stratum corneum (outermost layer of skin) and into the deeper skin layers. Patches containing arrays of microneedles, pointed structures having millimeter-scale heights and widths, can be used as delivery vehicles. In instances whereby the delivery of agents to a specific skin depth (e.g., dermal layer) is desirable, an applicator device provides a precise and repeatable means to deploy microneedle patches.

### SUMMARY

Devices and methods as described herein relate to applicators for intradermal microneedle array patches. These devices, methods, and techniques allow a user to apply an intradermal microneedle array patch to a skin region of an individual with a consistent and controllable penetration depth of the microneedles.

In some embodiments, a microneedle array patch applicator, comprises a shaft having an opening at a first end for affixing a microneedle array patch; a plunger positioned substantially within the shaft, the plunger having a first end positioned adjacent to the opening, wherein the first end of the plunger is of a size and shape to transfer force against the microneedle array patch, and the plunger having a holding structure at a second end of the plunger; a loading mechanism operably associated with the plunger within the shaft, the loading mechanism having a plunger load and a load spring, the plunger load positioned at a second end of the shaft, the load spring surrounding the plunger; and a force applicator, the force applicator having a holding interface adjacent to the second end of the plunger and a button protruding from the shaft, wherein at a loaded position, the holding structure is affixed to the holding interface and the load spring is compressed.

In some embodiments, a method of applying a microneedle array patch to a skin region of an individual, comprises affixing the microneedle array patch to a first end of a plunger of a microneedle array patch applicator, wherein the plunger is positioned within a shaft of the microneedle array patch applicator having an opening, and wherein the first end of the plunger is visible protruding from the opening at a neutral position; loading the plunger and the affixed microneedle array patch to a loaded position, wherein a holding structure associated with a second end of the plunger is thereby affixed to a holding interface of a force applicator positioned substantially within the shaft; positioning the opening against the skin region of the individual; operating the force applicator to release the plunger and thereby the holding structure being released from the holding interface and the microneedle array patch being inserted into the skin region of the individual.

In some embodiments, a microneedle array patch applicator, comprises a shaft having an opening at a first end for affixing a microneedle array patch; a plunger positioned substantially within the shaft, the plunger having a first end positioned adjacent to the opening, wherein the first end of the plunger is of a size and shape to transfer force against the microneedle array patch, and the plunger having a holding structure at a second end of the plunger; a plunger load operably associated with the plunger within the shaft, the plunger load positioned at a second end of the shaft; a load spring associated with the plunger load, the load spring surrounding the plunger; and a plunger release bar positioned substantially within the shaft, the plunger release bar having a holding interface adjacent to the second end of the plunger and a button protruding from the shaft, wherein at a loaded position, the holding structure is affixed to the holding interface and the load spring is compressed.

The foregoing summary is illustrative only and is not intended to be in any way limiting. Features from any of the disclosed embodiments can be used in combination with one another, without limitation. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows a perspective view of an applicator for intradermal microneedle patches in a neutral state.

FIGS. 2A and 2B show elevational views of the applicator of FIG. 1, with the applicator in the neutral state illustrated in FIG. 2A and a loaded state illustrated in FIG. 2B.

FIGS. 3A and 3B show cross-sectional views of the applicator of FIG. 1 in the neutral state.

FIGS. 4A-4D show cross-sectional views of the applicator of FIG. 1 in the loaded state.

FIG. 5 shows a flowchart illustrating a method of applying intradermal microneedle array patches to a skin surface.

FIG. 6 is a demonstration of the applicator of FIG. 1 being applied to a test skin surface.

### DETAILED DESCRIPTION

Some therapeutic agents, including some vaccines, can be delivered to an individual through a microneedle array patch attached to the surface of the skin. Microneedle patches include an array of small “microneedles” with a medicinal agent for application to the intradermal skin region of an individual. Generally, the microneedles are positioned, sized, and fabricated to be inserted from the surface of the skin to a distance between 0.1 mm and approximately 2 mm in order to deliver a medicinal agent to the vascular-rich intradermal, epidermal or upper dermal layers of the skin without impacting deeper nerve endings. Microneedles can be fabricated from a variety of materials as required by a specific use case and as compatible with particular medicinal agents, such as silicon, metal, ceramics, glass, carbohydrates, hydrogels and polymers. In some patches, the medicinal agent is coated on the exterior of the microneedles, and in others, the microneedles include hollow cores with medicinal agent in the interior. Some patches include microneedles fabricated from a material, such as a carbohydrate or biodegradable polymer, embedded with the medicinal agent, so the medicinal agent is released as the microneedles break down within the skin. Medicinal agents suitable for application by microneedle arrays include metformin, DNA and RNA oligonucleotides, vaccines, cancer therapeutics, peptides, insulin, and other hormones, and pain medicines such as lidocaine and meloxicam. See, for example, Waghule T et al., Microneedles: A Smart Approach and Increasing Potential for Transdermal Drug Delivery System, Biomedicine & Pharmacotherapy, Volume 109, 2019, pp. 1249-1258, which is herein incorporated by reference for all purposes.

The specific materials, size, shape and fabrication of a microneedle array patch for application to the skin of an individual depends on the type of medicinal agent(s) included for application as well as the therapeutic use case. Microneedle array patches for specific use cases include specific positioning, lengths, sizes and shapes of microneedles in the array. In addition, the specific positioning, lengths, sizes and shapes of the microneedles in an array will depend on the materials used to fabricate a microneedle array and associated patch. Aspects of microneedle array architecture included in specific microneedle array patches include pitch, or the distance between adjacent microneedles; height or length of the microneedles from base to tip; shape or geometry of the needles such as pyramidal, conical or triangular shapes; and the cross-section size of the base on the microneedle at the plane where it affixes to the application surface of the patch. These microneedle array architecture aspects are selected based on factors including the medicinal agent(s) integrated, delivery kinetics desirable for the medicinal agent(s), the materials of the microneedles and patch, and the fabrication techniques for the microneedles and patch.

Microneedle array patches need to be stored, handled, and applied correctly. The microneedles themselves can be fragile and patches must be handled carefully, including application with the appropriate force and direction to have the microneedles penetrate the skin to an appropriate degree with minimal skin damage. Microneedle arrays that have been handled improperly can include, for example, bent, broken or missing microneedles, which will not penetrate the skin to an appropriate depth for release of medical agent(s). Loss of functional microneedles from an array can result in lost efficacy of the patch and reduced transmission of the medicinal agent(s) to the skin region. Incorrect application can also result in skin damage from excessive force or direction of the force in application of a patch to the skin surface. Application of microneedle array patches should be done quickly and efficiently with the correct force applied at the correct angle to maximize efficacy with minimal skin damage.

This disclosure relates to devices and methods for application of microneedle array patches to the skin with appropriate penetration of the microneedles while maintaining structural integrity. The microneedle applicator devices and methods are designed to apply microneedle array patches to the skin surface with reliable and reproducible penetration into the skin layers as appropriate for the particular microneedle array patch being applied. The microneedle applicator devices and methods are designed for reproducible, consistent application of microneedle array patches to the skin of individuals in need thereof by people without extensive training or expertise, including medical personnel in medical clinics, emergency situations, health care workers serving routine and mass vaccination campaigns, and self-application by patients. The microneedle applicator devices and methods can result in more consistent application of microneedle array patches relative to manual application of microneedle array patches.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

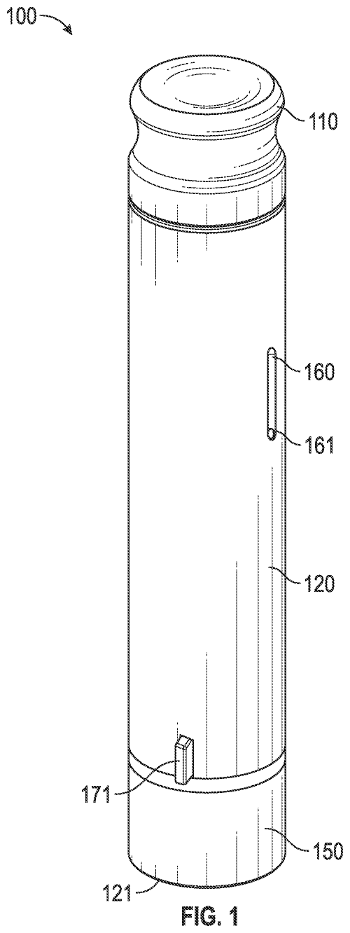

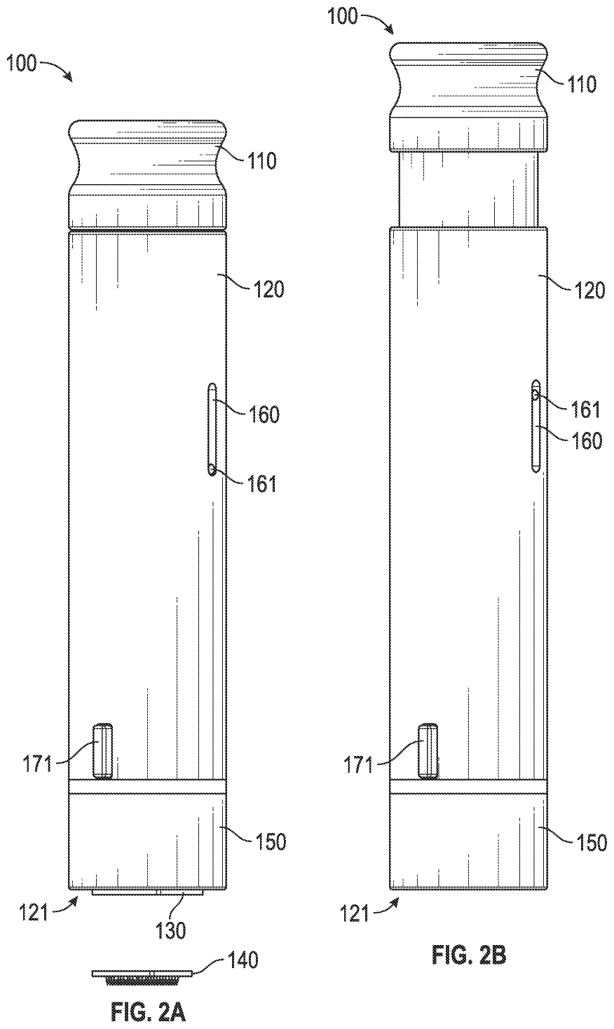

FIGS. 1, 2A and 2B illustrate external features of an applicator 100 for intradermal microneedle patches, according to some embodiments. The applicator 100 includes a shaft 120, a plunger load 110, a plunger 130 positioned within the shaft 120 configured to hold a microneedle array patch 140, and a button 171 for a user to release the microneedle array patch 140. The shaft 120 includes a first end with an opening 121 of a size and shape to permit movement of the plunger 130 within the interior of the shaft 120. As shown in FIG. 2A, a first end of the plunger 130 is visible protruding from opening 121 in a neutral state. The first end of the plunger 130 is not visible in the elevational view of FIG. 2B when the applicator 100 is in a loaded state. The first end of the plunger 130 is of a size and shape to put pressure on the top of the microneedle array patch 140 during application to the skin surface. The plunger load 110 is affixed at a second end of shaft 120 and operably associated with plunger 130 within the interior of shaft 120.

In the illustrated embodiment, the first end of the shaft 120 includes a removable flange 150 attached to assist with positioning the microneedle array patch 140 on a skin surface during the use of the applicator 100. The flange 150 is of a size, shape, and position that allows the plunger 130 to move within the interior of the shaft 120. With the flange 150 attached to the shaft 120, the first end of the plunger 130 is still visible in the neutral state, as shown in the elevational views. Some embodiments do not include a flange structure attached to the shaft 120.

In the illustrated embodiments, the applicator 100 includes an axial slot 160 on a sidewall of the shaft 120 and a pin 161 inside the axial slot 160 that can move up and down along the long axis of axial slot 160. Although not displayed in the current view, pin 161 is associated with a second end of the plunger 130 within the interior of the shaft 120. In the neutral state illustrated in FIG. 2A, pin 161 sits at a lower end of the axial slot 160. In some embodiments, the applicator 100 does not include the axial slot 160 or the pin 161. In some embodiments, the axial slot 160 is enclosed within the shaft 120, and the pin 161 moves along with the enclosed axial slot 160. Neither the axial slot 160 nor the pin 161 is visible externally. In some embodiments, pin 161 is a hook, a barb, a peg, or other similar structure that is associated with the plunger 130.

In the illustrated embodiment, the applicator 100 includes the button 171 adjacent to the first end of the shaft 120. The button 171 is part of a force applicator 170 (not entirely displayed in the current view) that releases the plunger 130 in a loaded state. The user can press the button 171, and then the microneedle array patch is released from the first end of the plunger 130 to the skin surface. In some embodiments, the button 171 may be a pin, a stud, a peg, a handle, a lever, or other structure that the user may apply force to release the plunger 130.

FIG. 2B illustrates the applicator 100 in a loaded state, according to some embodiments. Although not displayed in the current view, the plunger 130 is operably associated with the plunger load 110 within the interior of the shaft 120. The plunger load 110 can be pulled up along the long axis of the shaft 120 by the user. The plunger 130 is also pulled up in the same direction because of its association with the plunger load 110 within the shaft. The first end of the plunger 130 is therefore pulled inside the shaft 120 and not protruding from the opening 121 as shown in FIG. 2B. The plunger load 110 can be pushed down by the user without releasing the plunger 130 after the plunger 130 is properly affixed in the loaded state. In some embodiments, the user pushed down the plunger load 110 before applying the microneedle array patch 140 on the skin surface.

In the illustrated embodiments, the pin 161 is positioned at a higher end of the axial slot 160. The pin 161 can be a visual indicator of the loading status of the applicator. The plunger 130 is unloaded when the pin 161 is at the lower end of the axial slot 160 as illustrated in FIG. 2A. On the other hand, the plunger 130 is loaded when the pin 161 is at the higher end of the axial slot 160 as illustrated in FIG. 2B. Some embodiments do not have the visual indicator of the loading status.

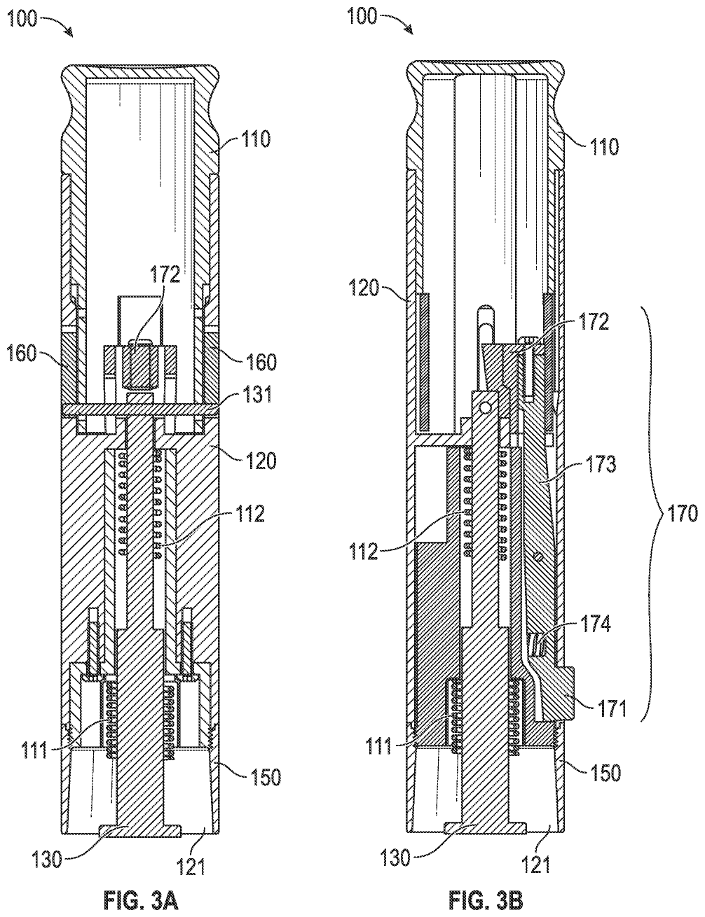

FIGS. 3A and 3B show cross-sectional views of the applicator 100 in the neutral state, according to some embodiments. The cross-sectional views illustrate the internal features of the applicator 100 in the neutral (unloaded) state. The plunger 130 is positioned within the hollow interior of the long axis of a shaft 120, with the first end of the plunger 130 protruding from an opening 121 in the first end of the shaft 120. The first end of the plunger 130 is of a size, shape and position relative to the applicator 100 as a whole to transmit force along its length to an adjacent intradermal microneedle array patch during use of the applicator 100. The interior of the shaft 120 is shaped and sized to permit the plunger 130 to move within the shaft 120 along the long axes of the shaft 120 and the plunger 130. The plunger load 110 is positioned at the second end of the shaft 120. A first end of the plunger load 110 is positioned within the interior of the shaft 120, and a second end of the plunger load 110 is protruding from the second end of the shaft 120. The plunger load 110 is operably associated with the plunger 130. One or more load springs associated with the plunger load 110 are surrounding the plunger 130.

In the illustrated embodiment, the applicator 100 has a first load spring 111 and a second load spring 112. The first load spring 111 and the second load spring 112 decouple the control over application velocity and holding pressure of the microneedle array patch during application. Application velocity is defined as the speed (cm/s) that a microneedle travels before insertion into the skin. Holding pressure (MPa) is defined as a constant force (N) applied to a microneedle after insertion into the skin per microneedle maximum cross-sectional area (cm²). The first load spring 111 and the second load spring 112 are adjustable to tune the application velocity and the holding pressure depending on the microneedle array architecture, such as the weight of the microneedle array patch, the geometry of the microneedles

In some embodiments, the applicator 100 only includes one load spring. The load spring can be positioned at any position along the long axis of the plunger 130. In some embodiments, the plunger load 110, the first load spring 111, and the second load spring 112 may form an integral loading mechanism. The loading mechanism is designed to move the plunger 130 within the shaft and affix the plunger 130 at a position where the user is able to control the release of the plunger 130.

In the illustrated embodiment, the removable flange 150 is affixed to the exterior of the shaft surrounding the opening 121. The flange 150 is substantially hollow and of an appropriate size, shape, and position to correspond with the first end of the plunger 130.

As shown in FIG. 3A, a second end of the plunger 130 includes a holding structure 131. In the embodiment illustrated, the holding structure 131 is shaped as a pin structure with both ends visible outside from the axial slot 160 (see FIGS. 1-2B). The pin 161, as shown in FIGS. 1-2B, is part of the holding structure 131. As the plunger 130 is moved up with the plunger load 110, the holding structure 131 is also moved up and affixed to a holding interface 172. The holding interface 172 is part of a force applicator 170, as shown in FIG. 3B.

In some embodiments, the holding structure 131 and the holding interface can be shaped as a hook, clip, barb, bristle, spike, arrowhead, curve, crook, or similar clasp structure so that the holding structure 131 and the holding interface 170 can interact and reversibly affix to each other. In some embodiments, the holding structure 131 is not visible outside. In some embodiments, the axial slot 160 may be an aperture that allows the movement of the holding structure 131 and is enclosed inside the shaft 120. The axial slot 160 may not be visible externally.

As shown in FIG. 3B, the force applicator 170 is positioned to apply force along the length of the plunger 130 when the holding structure 131 is released from the holding interface 172. The force applicator 170 is designed and fabricated to overcome the piercing and post-puncture resistive forces exerted by human skin during microneedle insertion. See Aggarwal and Johnson, Geometrical effects in mechanical characterizing of microneedle for biomedical applications, Sensors and Actuators B: Chemical, Volume 102, issue 2, 2004, pp. 226-234, which is herein incorporated by reference for all purposes. For example, depending on the embodiment, the force applicator 170 can be calibrated to apply a force in a range selected to overcome the theoretical piercing pressure of 3.18 MPa and post-puncture pressure of 1.6 MPa, depending on the situation and type of microneedle patch. See Aggarwal and Johnson, ibid. For example, depending on the embodiment, the applicator 100 can be calibrated to apply a known application velocity, e.g., 100 cm/s to 2000 cm/s. For example, depending on the embodiment, the applicator 100 can be calibrated to apply a known holding pressure per microneedle geometry, e.g., 0.25 MPa to 8 MPa. The particular amount of force required in a specific use case depends on factors including the weight of the moving mass, the depth required for therapeutic application of the microneedle array patch, the medicinal agent(s) included with the patch, the structure and position of the microneedles in the patch, and the materials used to fabricate the patch and microneedles.

In the illustrated embodiment, the force applicator 170 includes button 171, holding interface 172, a spring 174, and a plunger release bar 173. In some embodiments, the force applicator 170 can be modified for the application of different forces by changing the spring force.

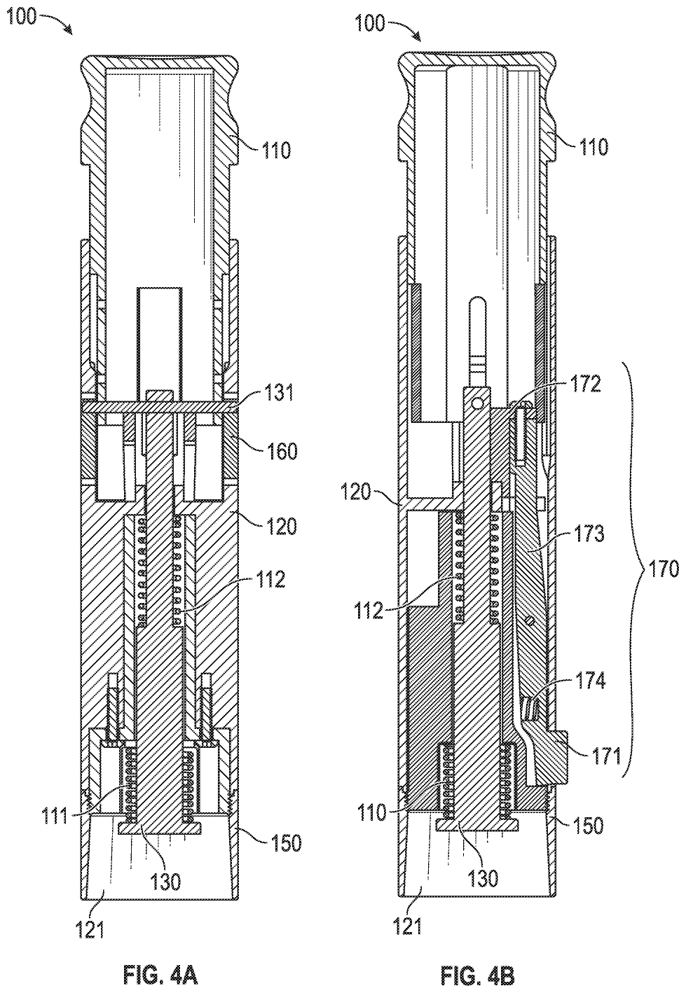

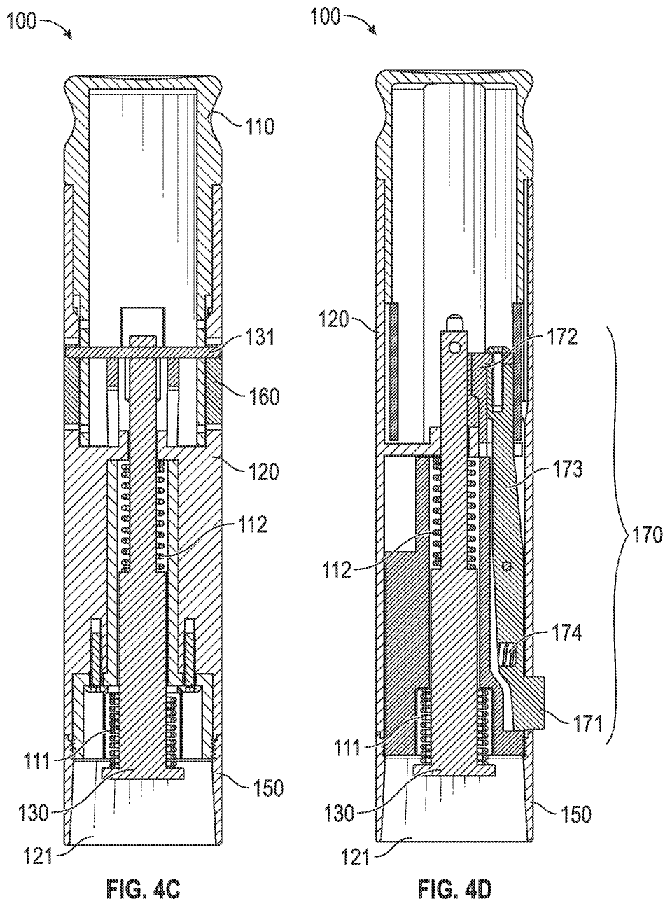

FIGS. 4A-4D shows cross-sectional views of the applicator 100 in the loaded state, according to some embodiments. The cross-sectional views illustrate internal features of the applicator 100 with components of the applicator 100 in a position with the holding structure 131 affixed to the holding interface 172. As shown in FIGS. 4A and 4B, the plunger load 110 is pulled up to load the plunger 130. FIGS. 4C and 4D illustrate the applicator 100 in cross-section as part of the plunger load 100 is pushed down into the shaft 120 after applicator 100 is loaded.

In the illustrated embodiment, the user has moved the plunger load 110 in a direction away from the second end of the shaft 120. The plunger 130 within the shaft 120 has correspondingly been moved so the holding structure 131 is reversibly engaged with the holding interface 172. The spring 174 affixed between the plunger release bar 174 and the button 171 creates tension on the position of the plunger release bar to maintain the holding interface 172 in place and retain the holding structure 131. The force applicator 170 correspondingly applies a force along the length of the plunger 130.

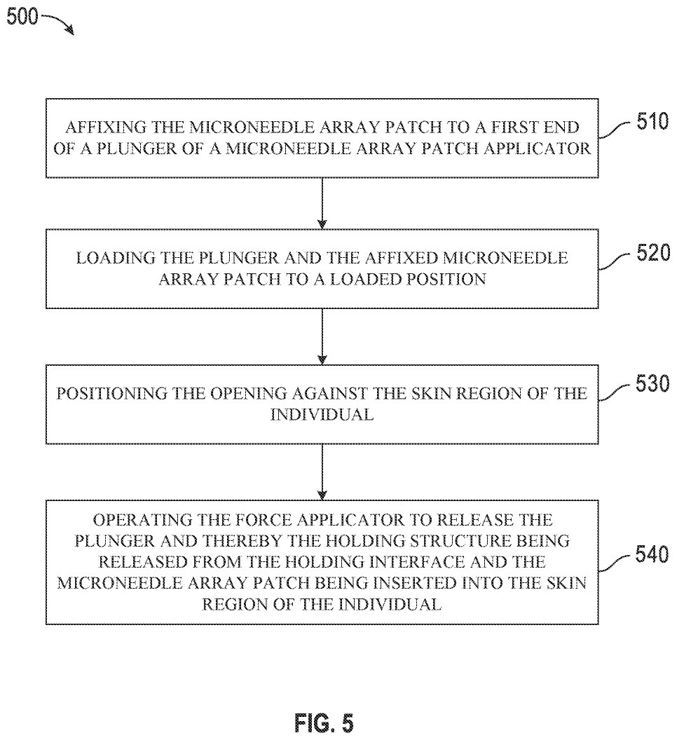

FIG. 5 illustrates a method of applying intradermal microneedle array patches to a skin surface of an individual, according to some embodiments. In general, the method begins by a person affixing the microneedle array patch 140 to the first end of the plunger 130 of the applicator 100 (step 510). The first end of the plunger 130 is of a size and shape that the microneedle array patches 140 can be pressed against and affixed to the plunger 130. The person loads the plunger 130 and the affixed microneedle array patch to a loaded position (step 520). The person pulls up the plunger load 110, and the plunger 130 correspondently is pulled up so that the holding structure 131 is reversibly engaging with the holding interface 172. The holding interface 172 is part of the force applicator 170 positioned substantially within the shaft 120. The next step is to position the opening 121 against the skin surface of an individual (step 530). In some embodiments, the applicator 100 has a removable flange 150 to assist the application of the microneedle array patch 140. The person operates the force applicator 170 to release the plunger 130, and thereby the holding structure 131 being released from the holding interface 174 and the microneedle array patch being inserted into the skin region of the individual (step 540). Release of the holding structure 131 permits the force applicator 170 to move the plunger 130 against the skin surface with an appropriate force to push the microneedles into the skin to a distance as needed for the specific patch. In some embodiments, the release of the holding structure 131 propels the plunger 130 at an application velocity in a range between 100 cm/s to 2000 cm/s. In some embodiments, the release of the holding structure 131 applies a constant pressure in a range between 0.25 MPa to 8 MPa. In some embodiments, the person can press the button 171 to release the plunger 130. The user can then remove the applicator, leaving the patch in position. The applicator can be reused as needed. Use of the applicator can provide reliable and consistent application of microneedle array patches to skin.

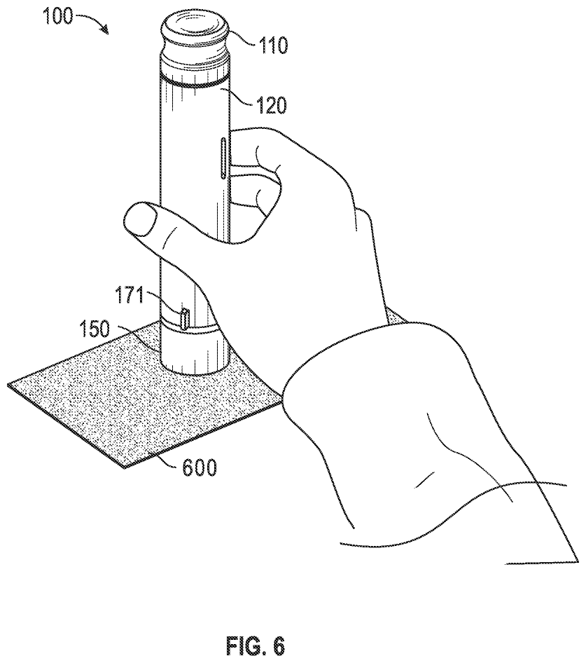

FIG. 6 illustrates the applicator 100 in a testing environment, according to some embodiments. In the illustrated example, an artificial skin 600 is used for testing purposes. The applicator 100 has been loaded with the microneedle array patch 140 attached to the plunger 130. A user's hand is shown gripping the shaft 120, with the user's thumb on the side closer to the button 171 and the second and third fingers positioned on the opposite side of the shaft 120. The button 171 is positioned so that the user's thumb can easily move to push the button 171 during the application of the microneedle array patch 140. The optional flange 170 can assist with positioning the microneedle array patch 140 against a region of the surface

The use of “including,” “comprising,” or “having” and variations thereof herein is meant to encompass the items listed thereafter and equivalents thereof as well as additional items. Unless specified or limited otherwise, the terms “affixed,” “associated,” “attached,” “connected,” “coupled” and “supported,” and variations thereof are used broadly and encompass both direct and indirect connections, supports, and couplings. It is to be understood that other embodiments may be utilized, and structural or logical changes may be made without departing from the scope of the present disclosure. Furthermore, terms such as “front,” “rear,” “top,” “bottom,” and the like are only used to describe elements as they relate to one another but are in no way meant to recite specific orientations of the apparatus, to indicate or imply necessary or required orientations of the apparatus, or to specify how the invention described herein will be used, mounted, displayed, or positioned in use.

Following are non-limiting examples of embodiments of the disclosure herein:

Example 1. A microneedle array patch applicator, comprising: a shaft having an opening at a first end for affixing a microneedle array patch; a plunger positioned substantially within the shaft, the plunger having a first end positioned adjacent to the opening, wherein the first end of the plunger is of a size and shape to transfer force against the microneedle array patch, and the plunger having a holding structure at a second end of the plunger; a loading mechanism operably associated with the plunger within the shaft, the loading mechanism having a plunger load and a load spring, the plunger load positioned at a second end of the shaft, the load spring surrounding the plunger; and a force applicator, the force applicator positioned substantially within the shaft to provide force along the length of the plunger, the force applicator having a holding interface adjacent to the second end of the plunger and a button on a sidewall of the shaft, wherein at a loaded position, the holding structure is affixed to the holding interface and the load spring is compressed.

Example 2. The applicator of Example 1, wherein the force applicator is calibrated to propel the plunger at an application velocity in a range between 100 cm/s to 2000 cm/s.

Example 3. The applicator of Example 1, wherein the force applicator is calibrated to release a constant force in a range between 0.25 MPa to 8 MPa.

Example 4. The applicator of Example 1, wherein the force applicator further comprises a plunger release bar connecting the holding interface and the button.

Example 5. The applicator of Example 1, wherein the force applicator further comprises a spring.

Example 6. The applicator of Example 1, further comprising a removable flange positioned at the first end of the shaft.

Example 7. The applicator of Example 1, wherein the first end of the plunger is visible protruding from the opening at a neutral position.

Example 8. The applicator of Example 1, further comprising a second load spring positioned adjacent to the first end of the plunger.

Example 9. The applicator of Example 1, further comprising an axial slot on a side wall of the shaft.

Example 10. The applicator of Example 9, wherein the holding structure is configured to move along a long axis of the axial slot.

Example 11. A microneedle array patch applicator, comprising: a shaft having an opening at a first end for affixing a microneedle array patch; a plunger positioned substantially within the shaft, the plunger having a first end positioned adjacent to the opening, wherein the first end of the plunger is of a size and shape to transfer force against the microneedle array patch, and the plunger having a holding structure at a second end of the plunger; a plunger load operably associated with the plunger within the shaft, the plunger load positioned at a second end of the shaft; a load spring associated with the plunger load, the load spring surrounding the plunger; and a plunger release bar positioned substantially within the shaft, the plunger release bar having a holding interface adjacent to the second end of the plunger and a button protruding from the shaft, wherein at a loaded position, the holding structure is affixed to the holding interface and the load spring is compressed.

Example 12. The applicator of Example 11, wherein the plunger release bar is calibrated to propel the plunger at an application velocity in a range between 100 cm/s to 2000 cm/s.

Example 13. The applicator of Example 11, wherein the plunger release bar is calibrated to release a constant pressure in a range between 0.25 MPa to 8 MPa.

Example 14. The applicator of Example 11, wherein the plunger release bar further comprises a spring.

Example 15. The applicator of Example 11, further comprising a removable flange positioned at the first end of the shaft.

Example 16. The applicator of Example 11, wherein the first end of the plunger is visible protruding from the opening at a neutral position.

Example 17. The applicator of Example 11, further comprising a second load spring positioned adjacent to the first end of the plunger.

Example 18. The applicator of Example 11, further comprising an axial slot on a side wall of the shaft.

Example 19. The applicator of Example 18, wherein the holding structure is configured to move along a long axis of the axial slot.

Example 20. A method of applying a microneedle array patch to a skin region of an individual, comprising: affixing the microneedle array patch to a first end of a plunger of a microneedle array patch applicator, wherein the plunger is positioned within a shaft of the microneedle array patch applicator having an opening, and wherein the first end of the plunger is visible protruding from the opening at a neutral position; loading the plunger and the affixed microneedle array patch to a loaded position, wherein a holding structure associated with a second end of the plunger is thereby affixed to a holding interface of a force applicator positioned substantially within the shaft; positioning the opening against the skin region of the individual; operating the force applicator to release the plunger and thereby the holding structure being released from the holding interface and the microneedle array patch being inserted into the skin region of the individual.

Example 21. The method of Example 20, wherein the plunger is operably associated with a loading mechanism, the loading mechanism having a load spring.

Example 22. The method of Example 20, wherein the plunger is operably associated with a loading mechanism, the loading mechanism having two load springs.

Example 23. The method of Example 20, further comprising: pushing down a plunger load into the shaft at the loaded position, wherein the plunger load is positioned at a second end of the shaft.

Example 24. The method of Example 20, wherein the microneedle array patch applicator comprises a removable flange positioned at the first end of the shaft.

Example 25. The method of Example 20, wherein the force applicator is calibrated to propel the plunger at an application velocity in a range between 100 cm/s to 2000 cm/s.

Example 26. The method of Example 20, wherein the force applicator is calibrated to release a constant pressure in a range between 0.25 MPa to 8 MPa.

Example 27. The method of Example 20, further comprising pressing a button positioned at a first end of the force applicator, wherein the button is protruding from the shaft.

Example 28. The method of Example 20, wherein loading the plunger and the affixed microneedle array patch to the loaded position comprises pulling a plunger load, wherein the plunger load is associated with the plunger.

Example 29. The method of Example 20, wherein the plunger is associated with a loading mechanism, the loading mechanism having a plunger load positioned at a second end of the shaft and a load spring surrounding the plunger.

Example 30. The method of Example 20, wherein the plunger is associated with a loading mechanism, the loading mechanism having a plunger load positioned at a second end of the shaft, a first load spring surrounding the first end of the plunger, and a second load spring surrounding the second end of the plunger.

What is claimed is:

1. A microneedle array patch applicator, comprising:
a shaft having an opening at a first end for affixing a microneedle array patch;
a plunger positioned substantially within the shaft, the plunger having a first end positioned adjacent to the opening, wherein the first end of the plunger is of a size and shape to transfer force against the microneedle array patch, and the plunger having a holding structure at a second end of the plunger;
a loading mechanism operably associated with the plunger within the shaft, the loading mechanism having a plunger load and a load spring, the plunger load positioned at a second end of the shaft, the load spring surrounding the plunger; and
a force applicator substantially within the shaft to provide force along the length of the plunger, the force applicator having a holding interface adjacent to the second end of the plunger and a button on a sidewall of the shaft,
wherein the holding structure is affixed to the holding interface and the load spring is compressed whereupon the plunger is at a loaded position.

2. The applicator of claim 1, wherein the force applicator is calibrated to propel the plunger at an application velocity in a range between 100 cm/s to 2000 cm/s.

3. The applicator of claim 1, wherein the force applicator is calibrated to release a constant pressure in a range between 0.25 MPa to 8 MPa.

4. The applicator of claim 1, wherein the force applicator further comprises a spring between the holding interface and the button.

5. The applicator of claim 1, further comprising a removable flange positioned at the first end of the shaft.

6. The applicator of claim 1, wherein the first end of the plunger is visible and protruding from the opening at a neutral position.

7. The applicator of claim 1, further comprising a second load spring positioned adjacent to the first end of the plunger.

8. A microneedle array patch applicator, comprising:
a shaft having an opening at a first end for affixing a microneedle array patch;
a plunger positioned substantially within the shaft, the plunger having a first end positioned adjacent to the opening, wherein the first end of the plunger is of a size and shape to transfer force against the microneedle array patch, and the plunger having a holding structure at a second end of the plunger;
a plunger load operably associated with the plunger within the shaft, the plunger load positioned at a second end of the shaft;
a load spring associated with the plunger load, the load spring surrounding the plunger; and
a plunger release bar positioned substantially within the shaft, the plunger release bar having a holding interface adjacent to the second end of the plunger and a button protruding from the shaft,
wherein the holding structure is affixed to the holding interface and the load spring is compressed whereupon the plunger is at a loaded position.

9. The applicator of claim 8, wherein the plunger release bar is calibrated to propel the plunger at an application velocity in a range between 100 cm/s to 2000 cm/s.

10. The applicator of claim 8, wherein the plunger release bar is calibrated to release a constant pressure in a range between 0.25 MPa to 8 MPa.

11. The applicator of claim 8, wherein the plunger release bar further comprises a spring.

12. The applicator of claim 8, further comprising a removable flange positioned at the first end of the shaft.

13. The applicator of claim 8, wherein the first end of the plunger is visible and protruding from the opening at a neutral position.

14. The applicator of claim 8, further comprising a second load spring positioned adjacent to the first end of the plunger.

15. A method of applying a microneedle array patch to a skin region of an individual, comprising:
affixing the microneedle array patch to a first end of a plunger of a microneedle array patch applicator, wherein the plunger is positioned within a shaft of the microneedle array patch applicator having an opening, and wherein the first end of the plunger is visible protruding from the opening at a neutral position;
loading the plunger and the affixed microneedle array patch to a loaded position, wherein a holding structure associated with a second end of the plunger is thereby affixed to a holding interface of a force applicator positioned substantially within the shaft;
positioning the opening against the skin region of the individual;
operating the force applicator to release the plunger and thereby the holding structure being released from the holding interface and the microneedle array patch being inserted into the skin region of the individual.

16. The method of claim 15, further comprising:
pushing down a plunger load into the shaft at the loaded position, wherein the plunger load is positioned at a second end of the shaft and associated with the plunger.

17. The method of claim 15, wherein the microneedle array patch applicator comprises a removable flange positioned at the first end of the shaft.

18. The method of claim 15, wherein the force applicator is calibrated to propel the plunger at an application velocity in a range between 100 cm/s to 2000 cm/s.

19. The method of claim 15, wherein the force applicator is calibrated to release a constant pressure in a range between 0.25 MPa to 8 MPa.

20. The method of claim 15, wherein the plunger is associated with a loading mechanism, the loading mechanism having a plunger load positioned at a second end of the shaft and a load spring surrounding the plunger.

21. The method of claim 15, wherein the plunger is associated with a loading mechanism, the loading mechanism having a plunger load positioned at a second end of the shaft, a first load spring surrounding the first end of the plunger, and a second load spring surrounding the second end of the plunger.
